(19) United States
(12) Patent Application Publication
(10) Pub. No.: US 2019/0004021 A1
(43) Pub. Date: Jan. 3, 2019

(54) ELECTRONIC DEVICE AND METHOD OF MONITORING SPECIFIC GAS

(71) Applicants: Fu Tai Hua Industry (Shenzhen) Co., Ltd., Shenzhen (CN); HON HAI PRECISION INDUSTRY CO., LTD., New Taipei (TW)

(72) Inventor: ZHI-HUI YU, Shenzhen (CN)

(21) Appl. No.: 15/689,574

(22) Filed: Aug. 29, 2017

(30) Foreign Application Priority Data
Jun. 30, 2017 (CN) 201710527210.X

(51) Int. Cl. G01N 33/00; G08B 21/14

(52) U.S. Cl. CPC G01N 33/0063; G08B 21/14; G01N 2033/0068; G01N 33/0073; G01N 33/004

(57) ABSTRACT

A method for monitoring of gas applied to an electronic device comprises a detecting device including activation of the detecting device to detect a gas concentration of a certain kinds of gas when one or more preset conditions are met. Gas concentration detected by the detecting device is obtained at preset time intervals. A preset warning prompt is transmitted when the obtained gas concentration is greater than or equal to a preset concentration.

### CROSS-REFERENCE TO RELATED APPLICATIONS

This application claims priority to Chinese Patent Application No. 201710527210.X filed on Jun. 30, 2017, the contents of which are incorporated by reference herein.

### FIELD

The subject matter herein generally relates to monitoring technology, and particularly to an electronic device and a method of monitoring harmful gas.

### BACKGROUND

Harmful gas such as carbon monoxide may exist everywhere. Too much harmful gas is a harmful effect to people's health.

### BRIEF DESCRIPTION OF THE DRAWINGS

Many aspects of the disclosure can be better understood with reference to the following drawings. The components in the drawings are not necessarily drawn to scale, the emphasis instead being placed upon clearly illustrating the principles of the disclosure. Moreover, in the drawings, like reference numerals designate corresponding parts throughout the several views.

FIG. 1 is a block diagram of one exemplary embodiment of an electronic device.

FIG. 2 illustrates a flow chart of one exemplary embodiment of a method of monitoring a concentration of harmful gas using the electronic device.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

The present disclosure, referencing the accompanying drawings, is illustrated by way of examples and not by way of limitation. It should be noted that references to “an” or “one” embodiment in this disclosure are not necessarily to the same embodiment, and such references mean “at least one.”

Furthermore, the term “module”, as used herein, refers to logic embodied in hardware or firmware, or to a collection of software instructions, written in a programming language, such as Java, C, or assembly. One or more software instructions in the modules can be embedded in firmware, such as in an EPROM. The modules described herein can be implemented as either software and/or hardware modules and can be stored in any type of non-transitory computer-readable medium or other storage device. Some non-limiting examples of non-transitory computer-readable media include CDs, DVDs, BLU-RAY, flash memory, and hard disk drives.

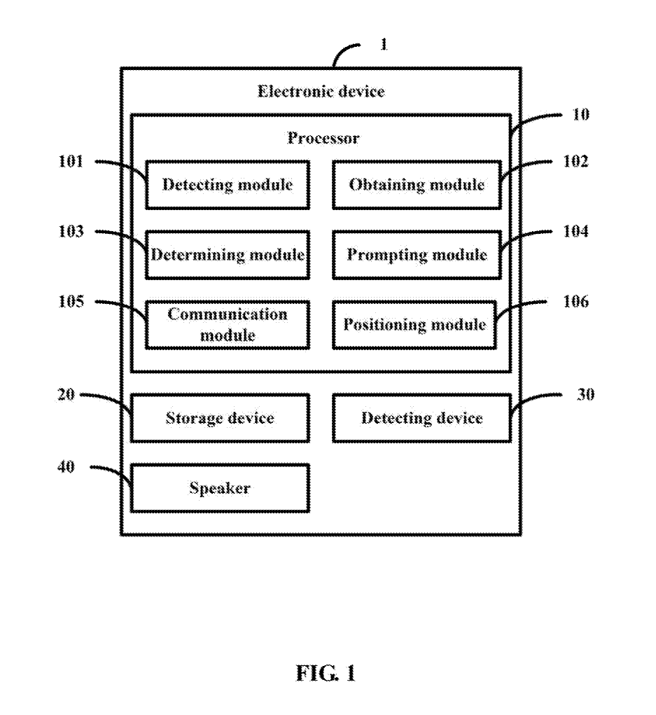

FIG. 1 is a block diagram of one exemplary embodiment of an electronic device 1. Depending on the embodiment, the electronic device 1 can include, but is not limited to, a processor 10, a storage device 20, a detecting device 30, and a speaker 40. The electronic device 1 can be a mobile phone, a tablet computer, or a personal digital assistant (PDA). The electronic device 1 can monitor a gas concentration of a certain kind of gas such as carbon monoxide or formaldehyde in a current environment.

In at least one exemplary embodiment, the detecting device 30 can be used to detect the gas concentration of the certain kind of gas. For example, the detecting device 30 may be a carbon monoxide sensor used for detecting the gas concentration of carbon monoxide, or may be a formaldehyde sensor used for detecting the gas concentration of formaldehyde. In other exemplary embodiments, the detecting device 30 may integrate with a plurality of sensors. The plurality of sensors can be used to detect different kinds of harmful gases. In at least one exemplary embodiment, the detecting device 30 can be configured in a front side of the electronic device 1, or in another side such as a left side or a right side of the electronic device 1 to facilitate to detect the gas concentration of the certain kind of gas.

The processor 10 can be a central processing unit (CPU), a microprocessor, or other data processor chip that performs functions of the electronic device 1.

In at least one exemplary embodiment, the storage device 20 can be internal storage such as a memory of the electronic device 1. In other exemplary embodiments, the storage device 20 can also be an external storage of the electronic device 1. For example, the storage device 20 can be a secure digital card, or a smart media card. The storage device 20 can be used to store data of the electronic device 1. For example, the storage device 20 stores the gas concentrations of the certain kinds of gas detected by the detecting device 30.

In at least one exemplary embodiment, the processor 10 can include, but is not limited to, a detecting module 101, an obtaining module 102, a determining module 103, a prompting module 104, a communication module 105, and a positioning module 106. In at least one exemplary embodiment, the modules 101-106 include computerized codes in the form of one or more programs that may be stored in the storage device 20. The computerized codes include instructions that can be executed by the processor 10. In other exemplary embodiments, the modules 101-106 may be instructions or firmware integrated in the processor 10.

In at least one exemplary embodiment, the detecting module 101 can activate the detecting device 30 to detect a gas concentration of the certain kinds of gas in a current environment.

In at least one exemplary embodiment, when the detecting device 30 is a carbon monoxide sensor, the gas concentration of carbon monoxide can be detected by the detecting device 30. When the detecting device 30 is a formaldehyde sensor, the gas concentration of formaldehyde can be detected by the detecting device 30.

In at least one exemplary embodiment, the detecting module 101 can activate the detecting device 30 to detect the gas concentration in response to user input. In other exemplary embodiments, the detecting module 101 can automatically activate the detecting device 30 to detect the local gas concentration when the electronic device 1 is located in a predetermined position. In at least one exemplary embodiment, the predetermined position can be indicated using a longitude and a latitude. In other exemplary embodiments, the detecting module 101 can automatically activate the detecting device 30 to detect the gas concentration in preset time periods (e.g., from 5 pm to 6 am every day). In other exemplary embodiments, the detecting module 101 can automatically activate the detecting device 30 to detect the gas concentration when the electronic device 1 is located in the predetermined position and a current time is within a preset time period (e.g., from 5 pm to 6 am every day).

In at least one exemplary embodiment, the detecting device 30 is a semiconductor gas sensor, i.e., the detecting device 30 is a sensor that is made of semiconducting material. In at least one exemplary embodiment, the semiconducting material of the detecting device 30 may be N-type semiconducting material such as SnO₂, or P-type semiconducting material such as NiO.

In other exemplary embodiments, the detecting device 30 can be an optical gas sensor, such as an infrared gas sensor.

The obtaining module 102 can obtain the gas concentration detected by the detecting device 30 at preset time intervals (e.g., every 30 seconds).

In at least one exemplary embodiment, the obtaining module 102 can obtain the gas concentration directly from the detecting device 30.

In other exemplary embodiments, the processor 10 can control the detecting module 101 to store the gas concentration detected by the detecting device 30 in the storage device 20, such that the obtaining module 102 can obtain the gas concentration from the storage device 20,

The determining module 103 can compare the obtained gas concentration with a preset concentration. In at least one exemplary embodiment, the preset concentration can be preset according to different environments. For example, when a user uses the electronic device 1 to detect the gas concentration of harmful gas such as carbon monoxide in a kitchen, the pre-set concentration is less than a critical concentration deemed harmful.

In at least one exemplary embodiment, the preset concentration can be preset in response to user input.

The prompting module 104 can transmit a preset prompt when the obtained gas concentration is greater than or equal to the preset concentration.

In at least one exemplary embodiment, the prompting module 104 can control the speaker 40 to play a preset audio, such that the user in the current environment is warned that the gas concentration of the certain kind of gas exceeds the preset concentration.

In at least one exemplary embodiment, the determining module 103 can further determine whether the preset prompt is shut off by a user in a preset time period (e.g., 1 minute, or 1.5 minutes). In at least one exemplary embodiment, the preset time period is calculated to begin from when the preset prompt is transmitted.

In at least one exemplary embodiment, when the preset prompt is not shut off by the user in the pre-set time period, the communication module 105 can generate an alarm, and can send the alarm to a preset list of people through Short Messaging Service (SMS). For example, the alarm may be a text message such as “the gas concentration of carbon monoxide in your kitchen exceeds the preset concentration, please take action immediately”. In other exemplary embodiments, the communication module 105 can automatically call the preset list of people.

In at least one exemplary embodiment, the positioning module 106 can obtain a current position of the electronic device 1. In at least one exemplary embodiment, the positioning module 106 can be a global positioning system (GPS). In at least one exemplary embodiment, the communication module 105 can send the current position to the preset list of people.

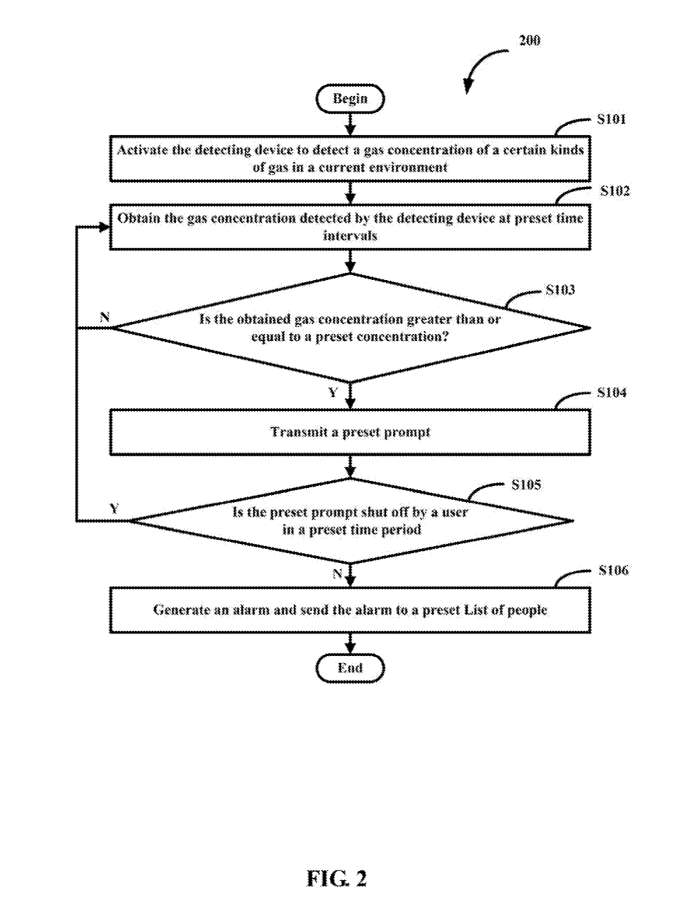

FIG. 2 illustrates a flowchart which is presented in accordance with an example embodiment. The exemplary method 200 is provided by way of example, as there are a variety of ways to carry out the method. The method 300 described below can be carried out using the configurations illustrated in FIG. 1, for example, and various elements of these figures are referenced in explaining exemplary method 200. Each block shown in FIG. 2 represents one or more processes, methods, or subroutines, carried out in the exemplary method 200. Additionally, the illustrated order of blocks is by example only and the order of the blocks can be changed according to the present disclosure. The exemplary method 200 can begin at block S101. Depending on the embodiment, additional steps can be added, others removed, and the ordering of the steps can be changed.

At block S101, the detecting module 101 can activate the detecting device 30 to detect a gas concentration of the certain kinds of gas in a current environment.

In at least one exemplary embodiment, when the detecting device 30 is a carbon monoxide sensor, the gas concentration of carbon monoxide can be detected by the detecting device 30. When the detecting device 30 is a formaldehyde sensor, the gas concentration of formaldehyde can be detected by the detecting device 30.

In at least one exemplary embodiment, the detecting module 101 can activate the detecting device 30 to detect the gas concentration in response to user input. In other exemplary embodiments, the detecting module 101 can automatically activate the detecting device 30 to detect the local gas concentration when the electronic device 1 is located in a predetermined position. In at least one exemplary embodiment, the predetermined position can be indicated using a longitude and a latitude. In other exemplary embodiments, the detecting module 101 can automatically activate the detecting device 30 to detect the gas concentration in preset time periods (e.g., from 5 pm to 6 am every day). In other exemplary embodiments, the detecting module 101 can automatically activate the detecting device 30 to detect the gas concentration when the electronic device 1 is located in the predetermined position and a current time is within a preset time period (e.g., from 5 pm to 6 am every day).

In at least one exemplary embodiment, the detecting device 30 is a semiconductor gas sensor, i.e., the detecting device 30 is a sensor that is made of semiconducting material. In at least one exemplary embodiment, the semiconducting material of the detecting device 30 may be N-type semiconducting material such as SnO₂, or P-type semiconducting material such as NiO.

In other exemplary embodiments, the detecting device 30 can be an optical gas sensor, such as an infrared gas sensor.

At block S102, the obtaining module 102 can obtain the gas concentration detected by the detecting device 30 at preset time intervals (e.g., every 30 seconds).

In at least one exemplary embodiment, the obtaining module 102 can obtain the gas concentration directly from the detecting device 30.

In other exemplary embodiments, the processor 10 can control the detecting module 101 to store the gas concentration detected by the detecting device 30 in the storage device 20, such that the obtaining module 102 can obtain the gas concentration from the storage device 20,

At block S103, the determining module 103 can compare the obtained gas concentration with a preset concentration. When the obtained gas concentration is greater than or equal to the preset concentration, the process goes to block S104. When the obtained gas concentration is less than the preset concentration, the process returns to block S102.

In at least one exemplary embodiment, the preset concentration can be preset according to different environment. For example, when a user uses the electronic device 1 to detect the gas concentration of harmful gas such as carbon monoxide in a kitchen, the pre-set concentration is less than a critical concentration deemed harmful.

In at least one exemplary embodiment, the preset concentration can be preset in response to user input.

At block S104, the prompting module 104 can transmit a preset prompt when the obtained gas concentration is greater than or equal to the preset concentration.

In at least one exemplary embodiment, the prompting module 107 can control the speaker 40 to play a preset prompt, such that the user in the current environment is warned that the gas concentration of the certain kinds of gas exceeds the preset concentration.

At block S105, the determining module 103 can further determine whether the preset prompt is shut off by a user in a preset time period (e.g., 1 minute, or 1.5 minutes). When the preset prompt is not shut off by the user in the pre-set time period, the process goes to block S106. When the preset prompt is shut off by the user in the pre-set time period, the process returns to block S102.

In at least one exemplary embodiment, the preset time period is calculated to begin from when the preset prompt is transmitted.

At block S106, when the preset prompt is not shut off by the user in the pre-set time period, the communication module 105 can generate an alarm, and can send the alarm to a preset list of people through Short Messaging Service (SMS). For example, the message may be a text message likes “the gas concentration of carbon monoxide in your kitchen exceeds the preset concentration, please take action immediately”. In other exemplary embodiments, the communication module 105 can automatically call the preset list of people

In at least one exemplary embodiment, the positioning module 106 can obtain a current position of the electronic device 1. In at least one exemplary embodiment, the positioning module 106 can be a global positioning system (GPS). In at least one exemplary embodiment, the communication module 105 can send the current position to the preset list of people.

It should be emphasized that the above-described embodiments of the present disclosure, including any particular embodiments, are merely possible examples of implementations, set forth for a clear understanding of the principles of the disclosure. Many variations and modifications can be made to the above-described embodiment(s) of the disclosure without departing substantially from the spirit and principles of the disclosure. All such modifications and variations are intended to be included herein within the scope of this disclosure and protected by the following claims.

What is claimed is:

1. An electronic device comprising:
a detecting device detecting a gas concentration of certain kinds of gas;
a processor;
a storage device storing computerized instructions, which when executed by the processor, cause the processor to:
activate the detecting device to detect the gas concentration when a preset condition is met;
obtain the gas concentration detected by the detecting device at preset time intervals; and
transmit a preset prompt when the obtained gas concentration is greater than or equal to a preset concentration.

2. The electronic device according to claim 1, wherein the preset condition comprises a condition that the electronic device is located in a predetermined position, a condition that a current time is within a first preset time period, or a combination thereof.

3. The electronic device according to claim 1, wherein the processor is further caused to:
determine whether the preset prompt is shut off by a user in a second preset time period; and
generate an alarm and send the alarm to a preset list of people.

4. The electronic device according to claim 3, wherein when the obtained gas concentration is greater than or equal to the preset concentration, the processor is further caused to:
obtain a current position of the electronic device; and
send the current position to the preset list of people.

5. The electronic device according to claim 1, wherein the detecting device is a carbon monoxide sensor that detects a gas concentration of carbon monoxide, or the detecting device is a formaldehyde sensor that detects a gas concentration of formaldehyde.

6. A gas monitoring method applied to an electronic device comprising a detecting device, the detecting device detecting a gas concentration of certain kinds of gas, the method comprising:
activate the detecting device to detect the gas concentration when a preset condition is met;
obtain the gas concentration detected by the detecting device at preset time intervals; and
transmit a preset prompt when the obtained gas concentration is greater than or equal to a preset concentration.

7. The gas monitoring method according to claim 6, wherein the preset condition comprises a condition that the electronic device is located in a predetermined position, a condition that a current time is within a first preset time period, or a combination thereof.

8. The gas monitoring method according to claim 6, further comprising:
determining whether the preset prompt is shut off by a user in a second preset time period; and
generating an alarm and send the alarm to a preset list of people.

9. The gas monitoring method according to claim 8, further comprising:
obtaining a current position of the electronic device when the obtained gas concentration is greater than or equal to the preset concentration; and
sending the current position to the preset list of people.

10. The gas monitoring method according to claim 6, wherein the detecting device is a carbon monoxide sensor that detects a gas concentration of carbon monoxide, or the detecting device is a formaldehyde sensor that detects a gas concentration of formaldehyde.
